(19) United States
(12) Patent Application Publication
(10) Pub. No.: US 2026/0118191 A1
(43) Pub. Date: Apr. 30, 2026

(54) ROLLING STRAIN SENSOR AND METHOD OF MANUFACTURING THE SAME

(71) Applicant: ELECTRONICS AND TELECOMMUNICATIONS RESEARCH INSTITUTE, Daejeon (KR)

(72) Inventors: Yun-Jeong KIM, Daejeon (KR); Hye-Jin KIM, Daejeon (KR); Sae-Rom SEO, Daejeon (KR)

(21) Appl. No.: 19/372,838

(22) Filed: Oct. 29, 2025

(30) Foreign Application Priority Data
Oct. 1, 2025 (KR) 10-2025-0143256
Oct. 31, 2024 (KR) 10-2024-0152369

(51) Int. Cl. G01L 1/14; A61B 5/00

(52) U.S. Cl. CPC G01L 1/142; A61B 5/6804; A61B 2562/0261; A61B 2562/125; A61B 2562/164

(57) ABSTRACT

Disclosed herein are a 3D-structured rolling strain sensor and a method of manufacturing the same. The 3D-structured rolling strain sensor includes a flexible substrate configured such that multiple films, each having a 2D electrode pattern formed thereon, are connected through tensile electrodes, and at least one elastomer sheet made of a stretchable material, wherein the flexible substrate is bonded to form a 3D structure while being rolled onto the at least one elastomer sheet, and the 3D structured rolling strain sensor is configured such that, when strain is externally applied, an overlapping area between upper and lower electrodes bonded to the at least one elastomer sheet varies, thereby changing a capacitance.

### CROSS REFERENCE TO RELATED APPLICATIONS

This application claims the benefit of Korean Patent Application Nos. 10-2024-0152369, October 31, 2024 and 10-2025-0143256, filed October 1, 2025, which are hereby incorporated by reference in their entireties into this application.

### BACKGROUND OF THE INVENTION

### 1. Technical Field

The present disclosure relates generally to a strain sensor having a three-dimensional (3D) structure (3D-structured strain sensor) and a technology of manufacturing the strain sensor, and more particularly to a high-sensitivity strain sensor technology for forming a 3D structure through a simple rolling process in which a 2D electrode substrate is wrapped with a flexible elastomer sheet, and measuring a subtle tensile change in a flexibly stretched region such as skin.

### 2. Description of Related Art

A strain sensor uses a principle that its resistance, capacitance, or electrical characteristics change in response to mechanical strain, such as externally applied tension or compression, and is used in various fields including structural deformation monitoring, wearable devices, robotics, and medical equipment. Various studies have been conducted to increase the sensitivity of such a strain sensor. Among the studies, one of processes that change a dimension (from 2D to 3D) involves a method utilizing an origami structure.

An origami strain sensor is a technology inspired by the traditional Japanese art of paper folding, in which a 2D planar structure is transformed into a 3D form to realize various physical properties. This structure is utilized as a sensor that may detect an electrical signal through mechanical deformation, and is being widely studied in fields where sensitivity and flexibility are of importance. In addition, these structural characteristics are advantageous for detecting mechanical deformation and may respond sensitively to tension and pressure applied from various directions. In other words, the origami strain sensor may realize a structurally complex shape and functions as a sensor that is sensitive to a change in the electrical signal.

However, a conventional origami strain sensor requires complex design and assembly processes to be converted into a 3D structure. These processes require a high degree of precision, and small errors or deformations that occur during a manufacturing process may reduce the signal reproducibility of the sensor, making it difficult to ensure reliability. This has a limitation in being applied to applications that require long-term monitoring or precise data collection. Further, due to a complex folding process, this structure tends to become excessively bulky or heavy, making it difficult to use in applications where miniaturization and light weight are essential, such as wearable devices, body-attachable sensors, and small electronic devices. Especially when portability and wearability should be considered, such increases in volume and weight present practical limitations.

### Prior Art Documents

### Patent Documents

(Patent Document 1) Korean Patent Application Publication No. 10-2022-0107980, Date of Publication: August 2, 2022 (Title: Pneumatic deployable variable stiffness soft link and production method of the same)

### SUMMARY OF THE INVENTION

Accordingly, the present disclosure has been made keeping in mind the above problems occurring in the prior art, and an object of the present disclosure is to provide a simple manufacturing process, which is advantageous for the mass production of a 3D-structured rolling strain sensor.

Another object of the present disclosure is to optimize the configuration of a 3D-structured rolling strain sensor by adjusting various parameters and to maintain stable performance even under repeated deformations by improving signal reproducibility compared to a conventional sensor.

A further object of the present disclosure is to provide a 3D-structured rolling strain sensor, which minimizes volume during a 3D structure formation process, thereby achieving miniaturization and weight reduction of the sensor, and enabling its application to various application fields such as a wearable device or a body-attachable sensor.

In accordance with an aspect of the present disclosure to accomplish the above objects, there is provided a three-dimensional (3D)-structured rolling strain sensor, including a flexible substrate configured such that multiple films, each having a two-dimensional (2D) electrode pattern formed thereon, are connected through tensile electrodes; and at least one elastomer sheet made of a stretchable material, wherein the flexible substrate is bonded to form a 3D structure while being rolled onto the at least one elastomer sheet, and the 3D-structured rolling strain sensor is configured such that, when strain is externally applied, an overlapping area between upper and lower electrodes bonded to the at least one elastomer sheet varies, thereby changing a capacitance.

The flexible substrate may be designed in consideration of a first parameter corresponding to a width, a length, a number of films, a length excluding an overlapping area between the films, and a tensile electrode angle.

The at least one elastomer sheet may be manufactured in consideration of a second parameter corresponding to a modulus, a sheet thickness, a number of sheets, and an arrangement method for the multiple sheets.

A strain stress or sensitivity to capacitance change may be adjusted based on the first parameter or the second parameter.

The strain stress may decrease as the tensile electrode angle decreases, and may increase as the tensile electrode angle increases.

As the sheet thickness decreases, an initial capacitance value may decrease, but the sensitivity to the capacitance change may increase.

When the at least one elastomer sheet is composed of multiple sheets having different moduli, strain stresses on facing surfaces in the sensor may be different from each other.

In accordance with another aspect of the present disclosure to accomplish the above object, there is provided a method of manufacturing a three-dimensional (3D)-structured rolling strain sensor, including designing a flexible substrate by connecting multiple films, each having a 2D electrode pattern formed thereon, through tensile electrodes; manufacturing at least one elastomer sheet from a stretchable material; and manufacturing a 3D-structured rolling strain sensor by rolling the flexible substrate onto the at least one elastomer sheet, wherein the 3D-structured rolling strain sensor is configured such that, when strain is externally applied, an overlapping area between upper and lower electrodes bonded to the at least one elastomer sheet varies, thereby changing a capacitance.

Designing the flexible substrate may include designing the flexible substrate in consideration of a first parameter corresponding to a width, a length, a number of films, a length excluding an overlapping area between the films, and a tensile electrode angle.

Manufacturing the at least one elastomer sheet may include manufacturing the at least one elastomer sheet in consideration of a second parameter corresponding to a modulus, a sheet thickness, a number of sheets, and an arrangement method for the multiple sheets.

A strain stress or sensitivity to capacitance change of the rolling strain sensor may be adjusted based on the first parameter or the second parameter.

The rolling strain sensor may be configured such that the strain stress decreases as the tensile electrode angle decreases, and increases as the tensile electrode angle increases.

The rolling strain sensor may be configured such that, as the sheet thickness decreases, an initial capacitance value decreases, but the sensitivity to the capacitance change increases.

The rolling strain sensor may be configured such that, when the at least one elastomer sheet is composed of multiple sheets having different moduli, strain stresses on facing surfaces in the sensor are different from each other.

### BRIEF DESCRIPTION OF THE DRAWINGS

The above and other objects, features and advantages of the present disclosure will be more clearly understood from the following detailed description taken in conjunction with the accompanying drawings, in which:

FIGS. 1 to 3 are diagrams illustrating an example of the configuration and manufacturing process of a 3D-structured rolling strain sensor according to the present disclosure;

FIGS. 4 and 5 are diagrams illustrating an example of a flexible substrate on which a 2D electrode pattern is formed, according to the present disclosure;

FIG. 6 is a diagram illustrating a 3D-structured rolling strain sensor according to an embodiment of the present disclosure;

FIG. 7 is a diagram illustrating types of a strain sensor according to the thickness and shape of an elastomer sheet in accordance with the present disclosure;

FIG. 8 is a diagram illustrating the measurement principle of a rolling strain sensor according to an embodiment of the present disclosure;

FIGS. 9 to 13 are diagrams illustrating the results of FEA analysis of a strain stress change according to the modulus of the elastomer sheet in accordance with the present disclosure;

FIGS. 14 and 15 are diagrams illustrating an example of a tensile electrode angle and the results of FEA analysis of a stress change corresponding thereto, according to the present disclosure;

FIGS. 16 and 17 are diagrams illustrating an example of images of the rolling strain sensor before and after stretching, and the results of a capacitance change according to a change in elastomer thickness caused by stretching, in accordance with the present disclosure; and

FIG. 18 is a flowchart illustrating a method of manufacturing a 3D-structured rolling strain sensor according to an embodiment of the present disclosure.

### DESCRIPTION OF THE PREFERRED EMBODIMENTS

The present disclosure will be described in detail below with reference to the accompanying drawings. Repeated descriptions and descriptions of known functions and configurations which have been deemed to make the gist of the present disclosure unnecessarily obscure will be omitted below. The embodiments of the present disclosure are intended to fully describe the present disclosure to a person having ordinary knowledge in the art to which the present disclosure pertains. Accordingly, the shapes, sizes, etc. of components in the drawings may be exaggerated to make the description clearer.

In the present specification, each of phrases such as “A or B”, “at least one of A and B”, “at least one of A or B”, “A, B, or C”, “at least one of A, B, and C”, and “at least one of A, B, or C” may include any one of the items enumerated together in the corresponding phrase, among the phrases, or all possible combinations thereof.

Hereinafter, embodiments of the present disclosure will be described in detail with reference to the attached drawings.

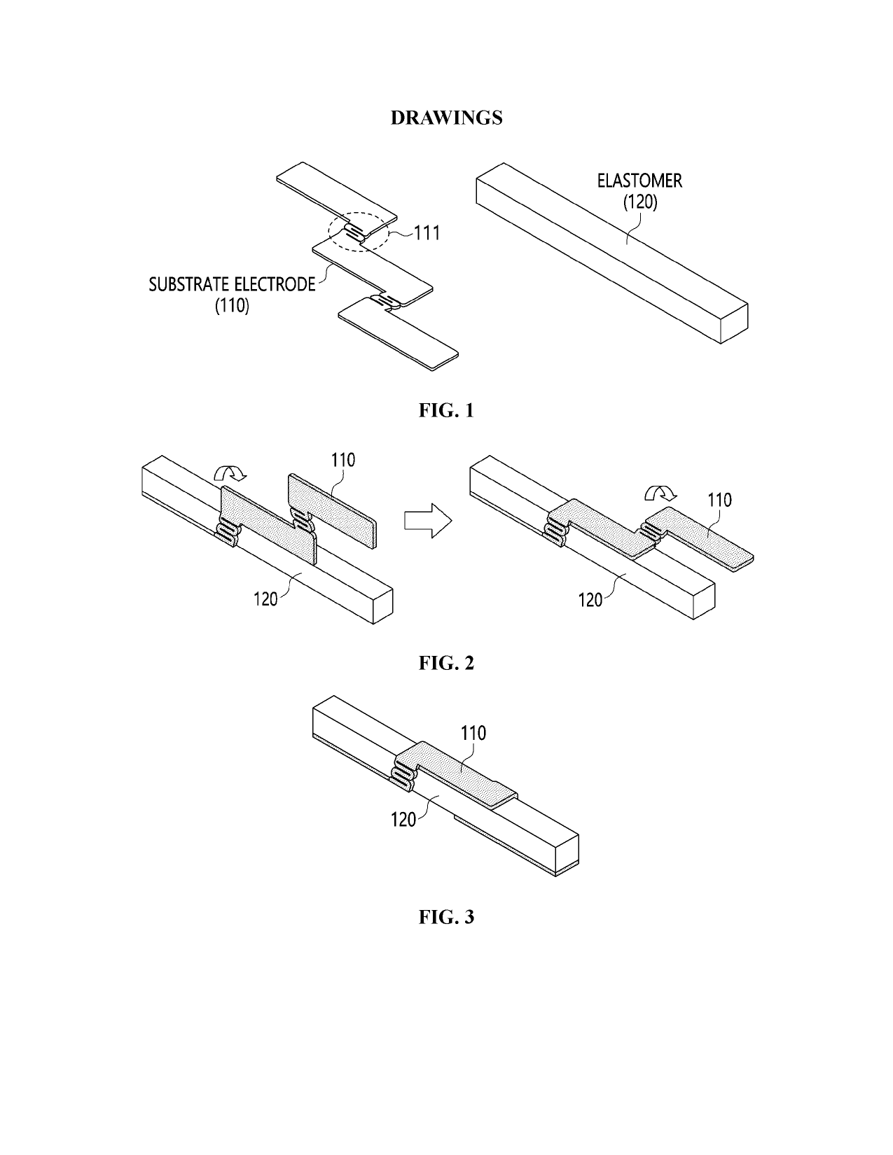

FIGS. 1 to 3 are diagrams illustrating an example of the configuration and manufacturing process of a 3D-structured rolling strain sensor according to the present disclosure.

Referring to FIG. 1, the 3D-structured rolling strain sensor according to the present disclosure may include a flexible substrate 110 in which multiple films having 2D electrode patterns are connected through tensile electrodes 111, and at least one elastomer sheet 120 made of a stretchable material.

In this case, the flexible substrate 110 may correspond to a substrate capable of 2D electrode patterning and sensor integration. For example, the flexible substrate 110 may be formed of materials such as polyimide (PI), polypropylene (PP), polyurethane (PU), polyethylene (PE), or polystyrene (PS), and may correspond to a composite material (FCCL) in which a copper layer is laminated on a PI substrate. In addition, the flexible substrate 110 may be processed using scissors or a knife, and the substrate with a complex pattern or a high-resolution pattern may be manufactured through a UV laser.

In this case, the elastomer sheet 120 may be made of a stretchable material such as PDMS, Ecoflex, or Clear-flex, and may be made in the form of a film or a porous structure to have a different modulus. Further, the elastomer sheet 120 may be manufactured with various materials and forms having different moduli, for example, by adding a conductive filler to increase a dielectric constant.

In this case, the elastomer sheet 120 may be adjustable in thickness, and one or multiple sheets may be used.

In this case, the flexible substrate 110 may be bonded to form a 3D structure while being rolled onto at least one elastomer sheet 120.

For example, as illustrated in FIG. 2, a thin adhesive or adhesive film may be attached to each of n films forming the flexible substrate 110 to be bonded to the elastomer sheet 120 in a wrapping manner. In this case, each of portions of the flexible substrate 110 connected by the tensile electrode 111 may correspond to a film portion with an electrode pattern. In this manner, all of the n films may be rolled and bonded to the elastomer sheet 120 to manufacture the 3D-structured rolling strain sensor, as illustrated in FIG. 3. In this case, although a single elastomer sheet 120 is used in FIG. 3, the elastomer sheet may be employed in various forms, such as an integrated type using a single sheet or a divided type using multiple sheets.

In this case, the flexible substrate 110 may be designed in consideration of a first parameter corresponding to a width, a length, a number of the films, a length excluding an overlapping area between the films, and a tensile electrode angle.

In this case, the tensile electrode 111 may have various structures including a structurally stretchable horseshoe structure, and may be formed of a composite material including a stretchable material.

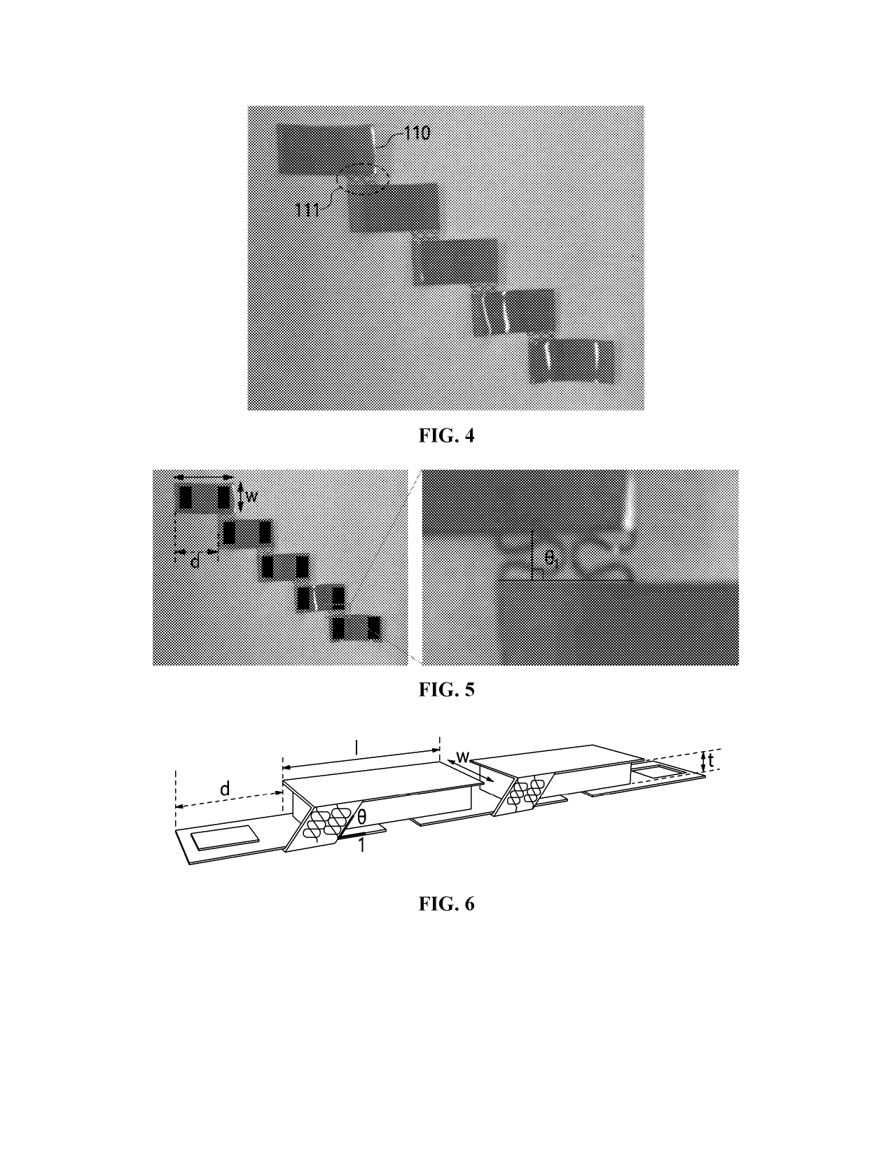

For example, referring to FIGS. 4 and 5, the flexible substrate 110 on which the 2D electrode pattern is formed may be designed to adjust parameters such as a width W, a length I, a number n of films, a length d excluding the overlapping area between the films, and a tensile electrode angle θ₁. By adjusting these parameters, the overall length and width of the sensor may be optimized.

That is, referring to FIG. 6, the width W of the flexible substrate 110 illustrated in FIG. 5 may determine the width of the sensor as illustrated in FIG. 6, and the length I of the flexible substrate 110 illustrated in FIG. 5, the number n of the films, and the length d excluding the overlapping area between the films may determine the overall length of the sensor as illustrated in FIG. 6.

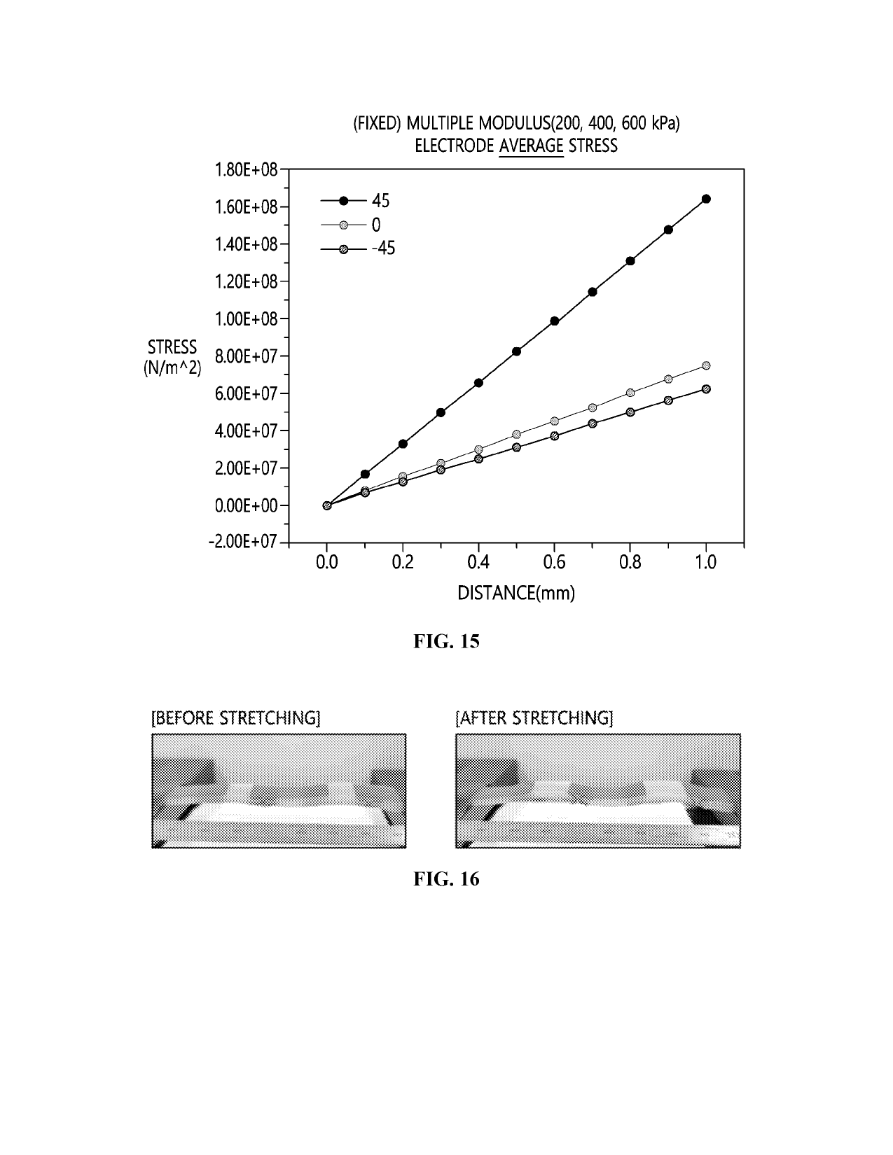

In this case, the tensile electrode angle θ₁ of the flexible substrate 110 illustrated in FIG. 5 may determine the tensile force or sensitivity of the sensor, and a detailed description related thereto will be provided later with reference to FIGS. 14 and 15.

In this case, at least one elastomer sheet 120 may be manufactured in consideration of a second parameter corresponding to the modulus, the thickness of the sheet, the number of sheets, and the arrangement method of the multiple sheets.

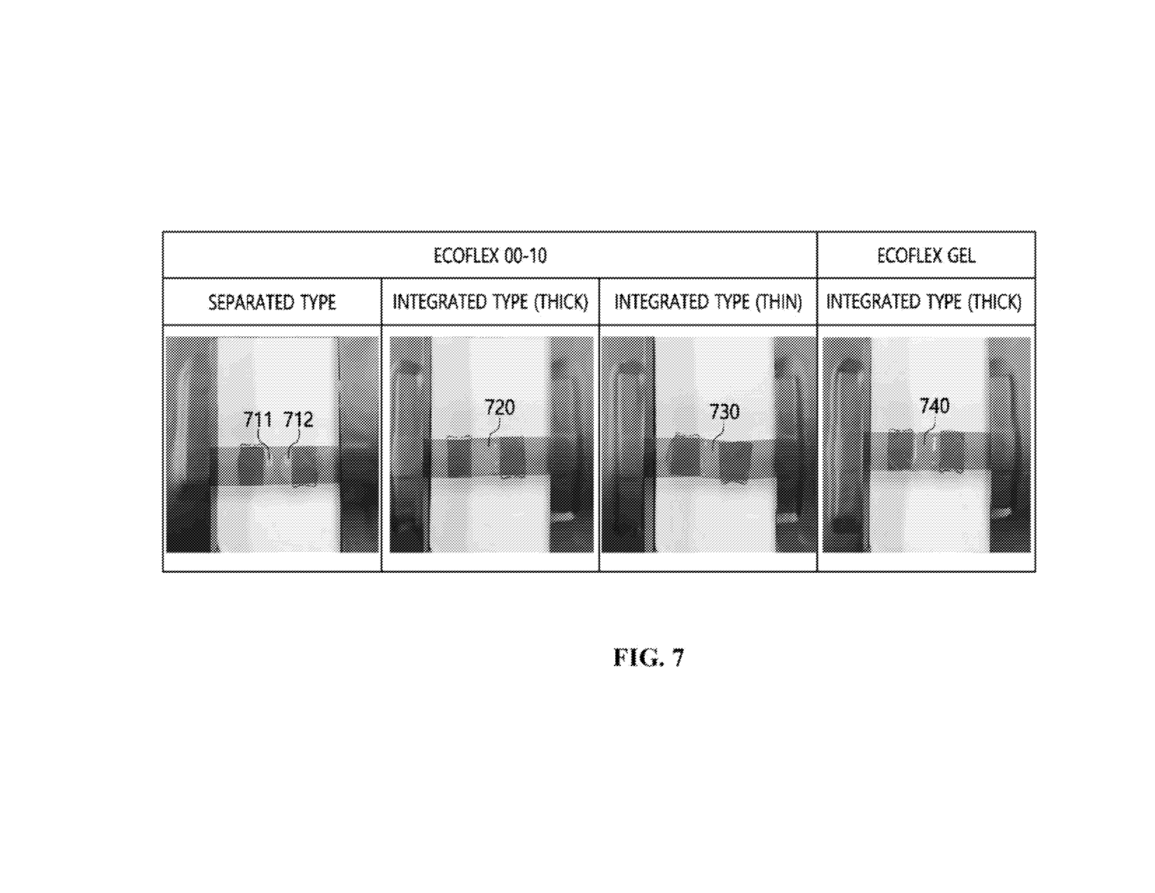

For example, FIG. 7 illustrates rolling strain sensors manufactured using various elastomer sheets. Referring to FIG. 7, the elastomer sheets may be manufactured and used in various forms depending on the intended application of the sensor, such as a separated type utilizing separated elastomer sheets 711 and 712 made of Ecoflex 00-10 material, an integrated type utilizing a relatively thick elastomer sheet 720 made of Ecoflex 00-10 material, an integrated type utilizing a relatively thin elastomer sheet 730 made of Ecoflex 00-10 material, and an integrated type utilizing a relatively thick elastomer sheet 740 made of Ecoflex gel material.

In this case, when an external strain is applied to the rolling strain sensor, the capacitance may change as the overlapping area between upper and lower electrodes bonded to at least one elastomer sheet 120 varies.

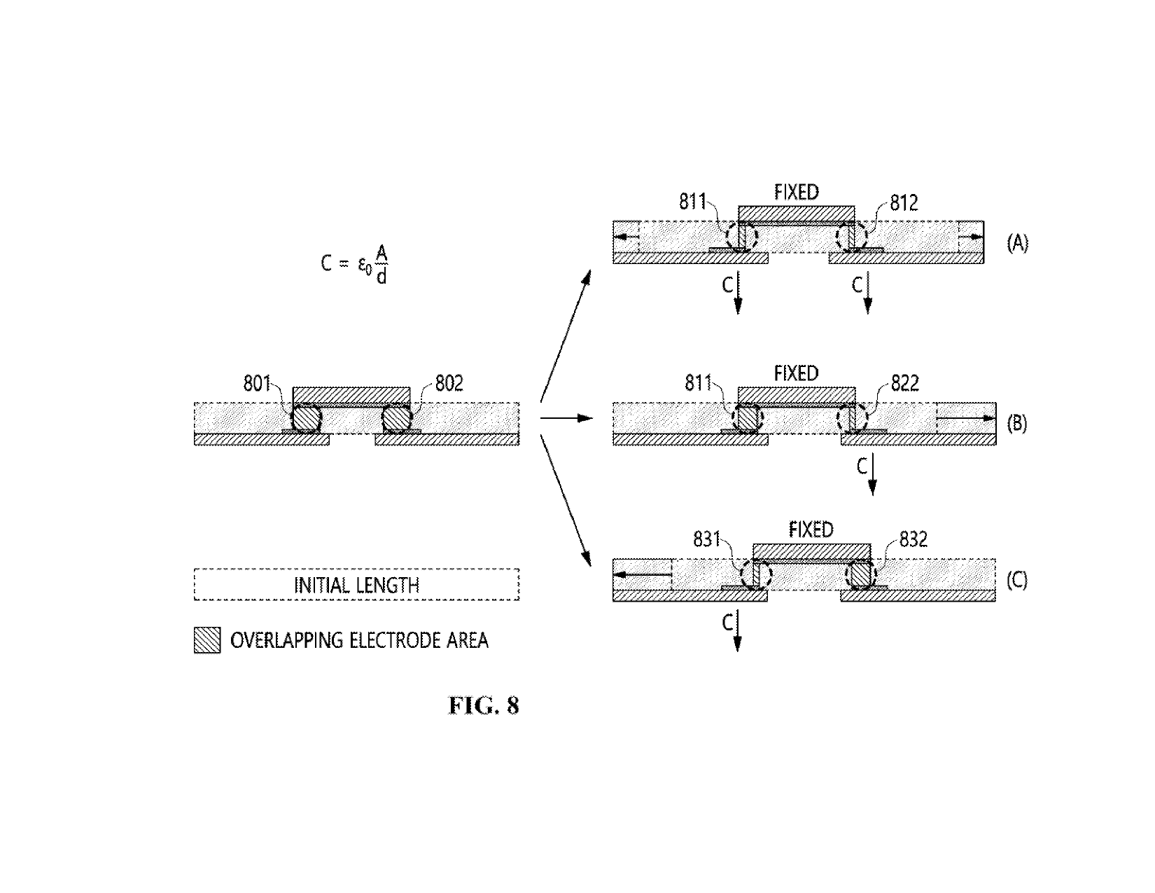

For example, the 3D-structured rolling strain sensor according to the present disclosure may operate based on the principle of measuring the difference in capacitance caused by changes in the thickness and area of the upper and lower electrodes when the sensor is stretched or contracted, as illustrated in FIG. 8. That is, the sensor shown on the left side of FIG. 8 represents a default state in which no stretching or contraction occurs, while three sensor states A, B, and C shown on the right side of FIG. 8 represent states stretched on both sides, to the right, and to the left, respectively. Comparing overlapping electrode areas 801 and 802 between the upper and lower electrodes when the sensor is in the default state with overlapping electrode areas 811, 812, 821, 822, 831, and 832 between the upper and lower electrodes when the sensor is in the states A, B, and C, it can be observed that the area decreases as the sensor is stretched. As the overlapping electrode area decreases, the capacitance also decreases, so the sensor may measure the degree of stretching based on the difference in the reduced capacitance.

In this case, tension distribution may be measured through an array electrode channel, and the capacitance may also vary with a change in the thickness of the elastomer sheet, allowing the degree of stretching to be measured.

Thus, both the change in the overlapping area between the upper and lower electrodes and the change in the thickness of the elastomer sheet may be used to calibrate the measured stretching degree, or may be applied to artificial intelligence learning to measure the stretching degree more accurately.

In this case, the strain stress or the sensitivity to capacitance change may be adjusted based on the first parameter and the second parameter.

Therefore, in the present disclosure, a customized strain sensor may be manufactured by adjusting various parameters.

In this case, when at least one elastomer sheet is composed of multiple sheets having different moduli, strain stresses on the facing surfaces in the sensor may differ from each other.

That is, by adjusting the modulus conditions of the elastomer sheet, portions with higher and lower strain stresses may be set.

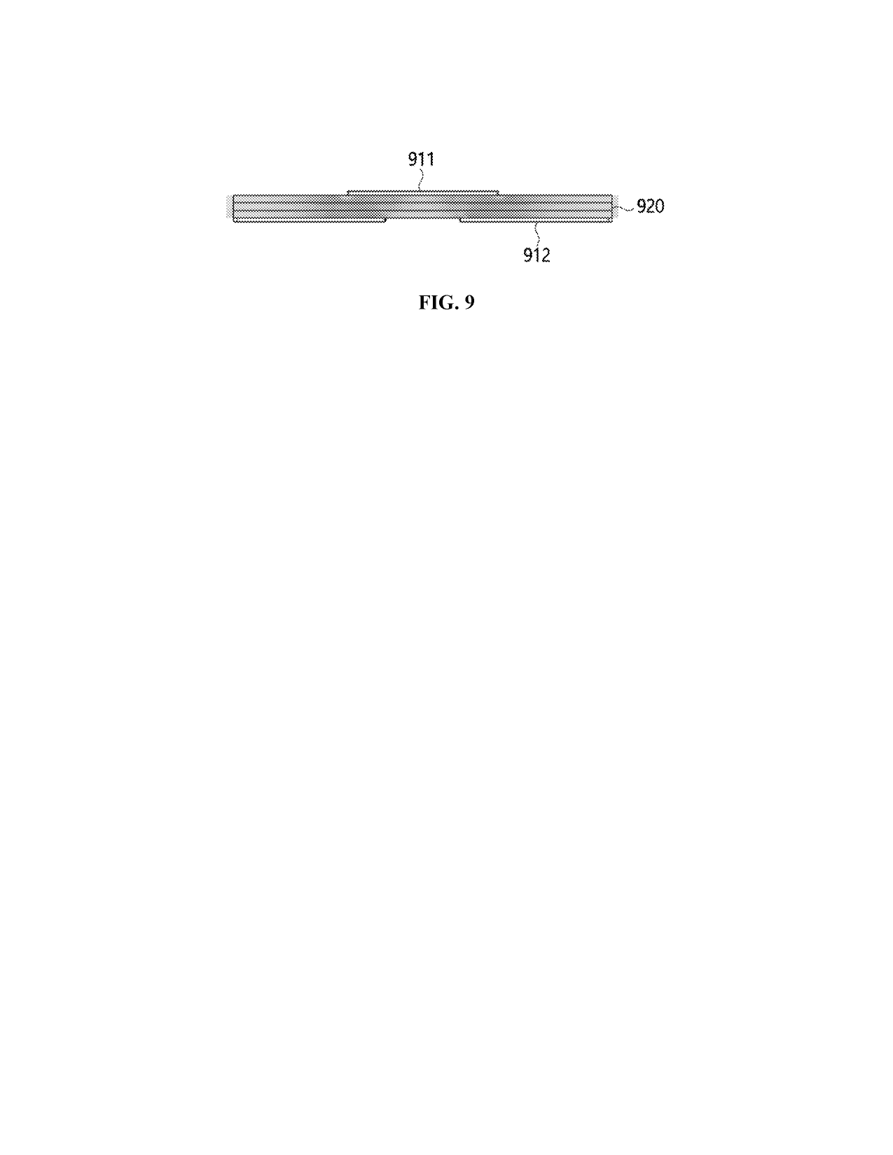

Hereinafter, with reference to FIGS. 9 to 13, a detailed description will be given of how the strain stress differs between a case where the elastomer sheet is configured with a single modulus of 600 kPa and a case where the elastomer sheet is configured with three moduli of 600 kPa, 400 kPa, and 200 kPa.

In this case, as illustrated in FIG. 9, the sensor composed of the elastomer sheet with three moduli is formed in a structure in which the three moduli are stacked in layers, with a top layer having the modulus of 600 kPa, a middle layer having the modulus of 400 kPa, and a bottom layer having the modulus of 200 kPa.

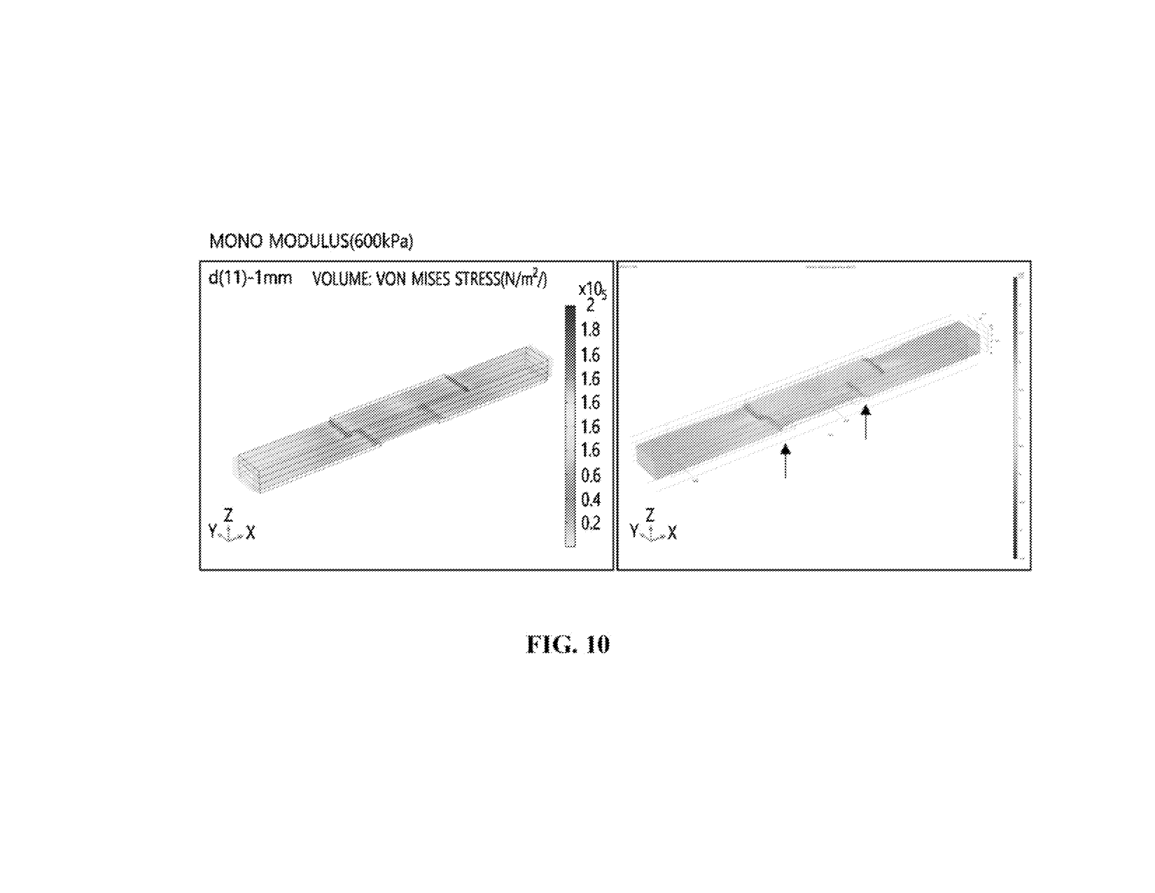

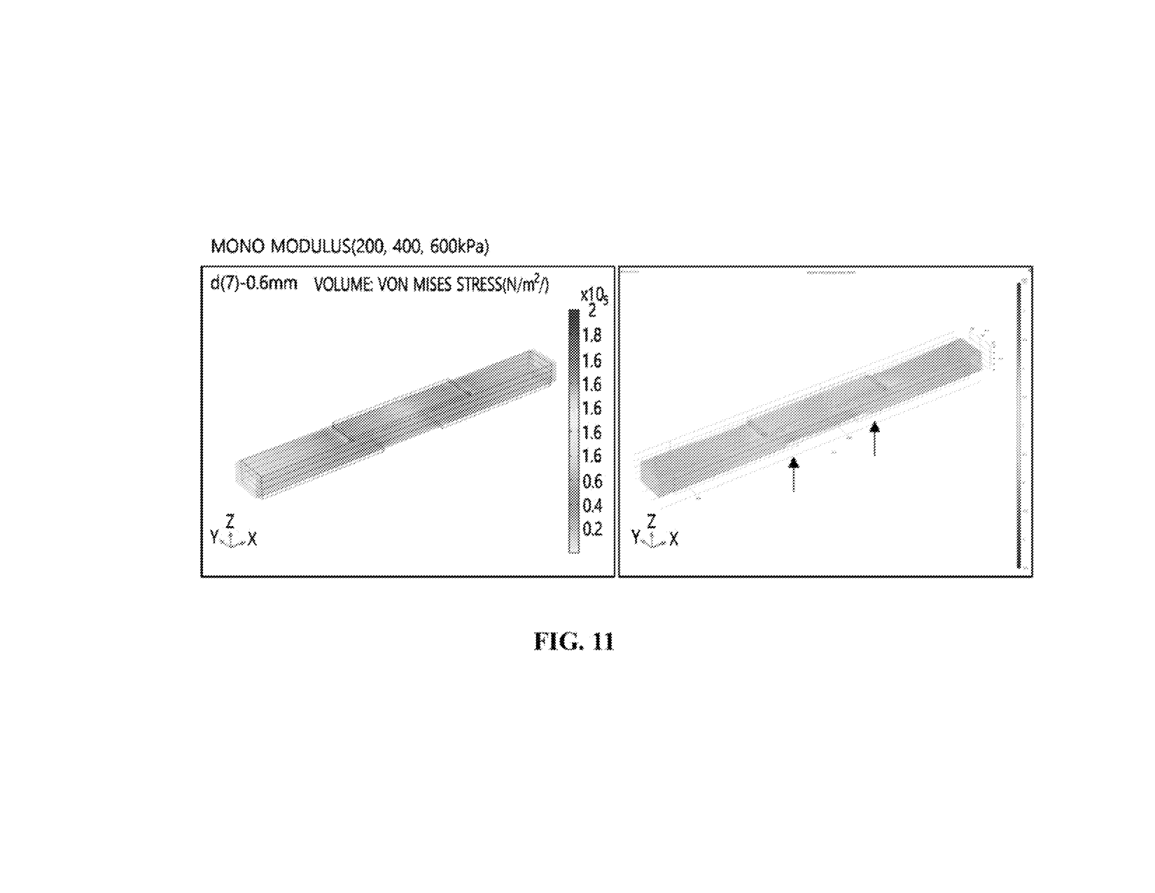

In this case, FIG. 10 shows the result of measuring the strain stress when the elastomer sheet is configured with a single modulus of 600 kPa, and FIG. 11 shows the result of measuring the strain stress when the elastomer sheet is configured with three moduli of 600 kPa, 400 kPa, and 200 kPa.

When comparing the two results, it can be seen that, when the elastomer sheet is configured with the single modulus of 600 kPa, the strain stress is high at both ends of an upper substrate 911 and a lower substrate 912. In contrast, it can be seen that, when the elastomer sheet is configured with three moduli of 600 kPa, 400 kPa, and 200 kPa, the upper substrate 911 exhibits a higher strain stress distribution at both ends than the lower substrate 912. That is, it can be seen that the larger the modulus, the greater the strain stress, which may mean that the sensor is more likely to break. Therefore, the sensor configured with the single modulus of 600 kPa is more likely to break than the sensor configured with three moduli of 600 kPa, 400 kPa, and 200 kPa.

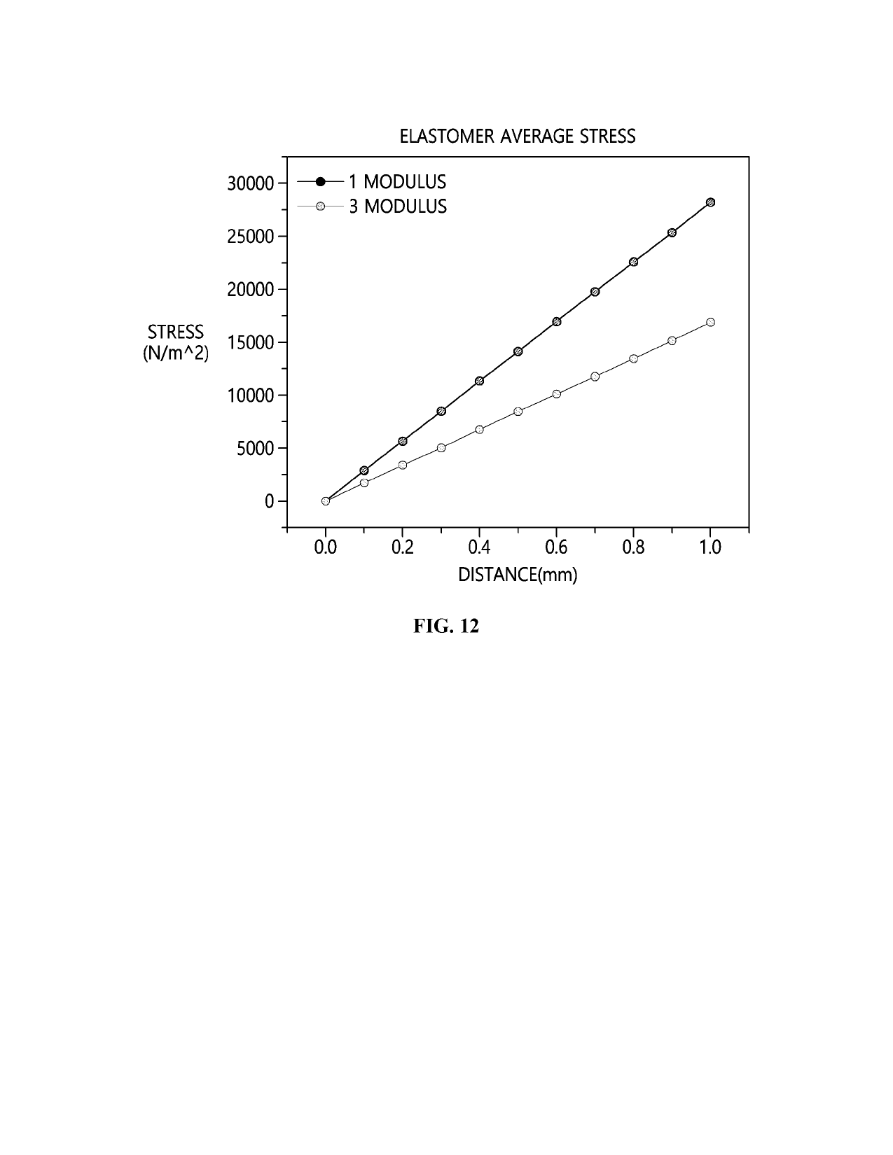

FIG. 12 shows the result of comparing such stresses, indicating that the sensor configured with three moduli exhibits a reduced stress level for the overall strain compared to the sensor configured with the single modulus. This may mean that the sensor is less likely to break.

If these results are applied to the strain sensor for measuring the degree of skin tension, it may be advantageous to place a high-modulus elastomer sheet on the opposite side (upper side) of the skin as it needs to remain fixed, and to place a low-modulus elastomer sheet on the skin side (lower side) as it requires a high degree of freedom under tension. In other words, the sensor may be designed to create a large stress difference between the opposite side of the skin and the skin side.

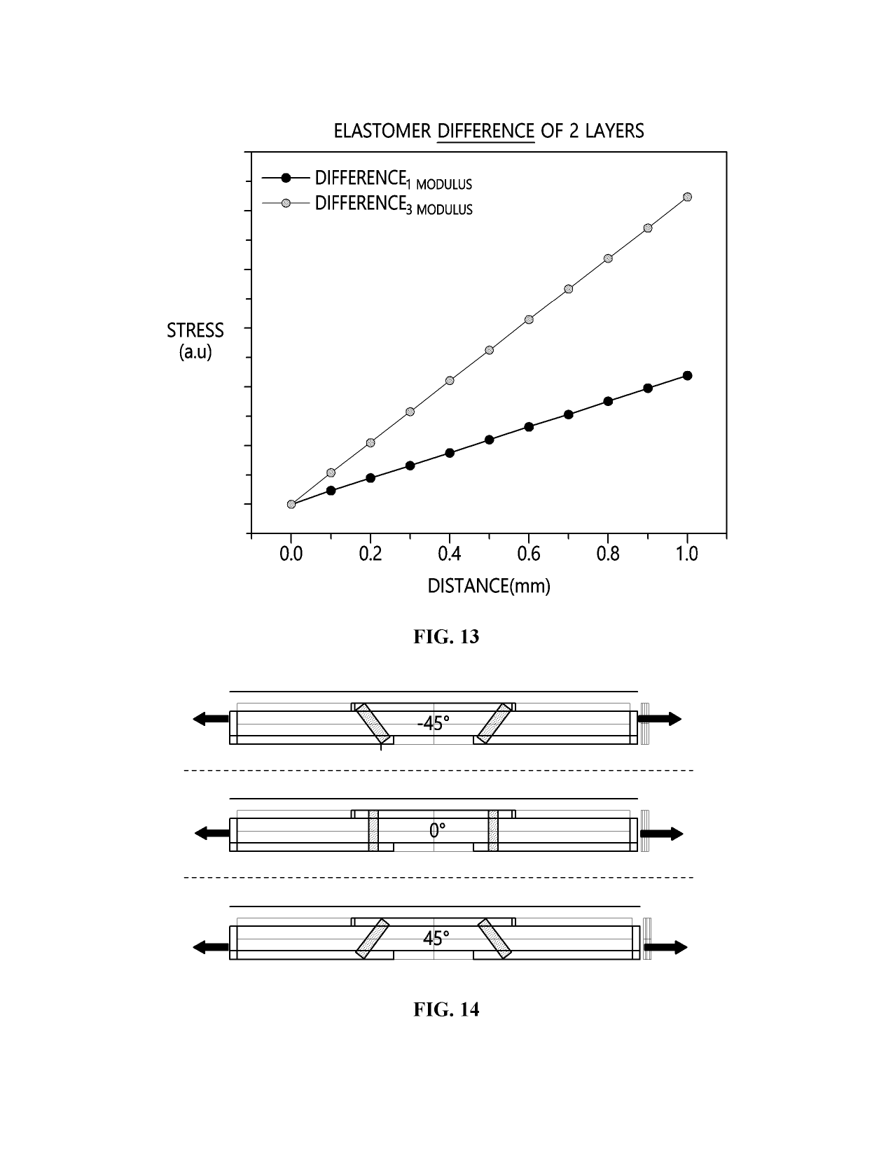

FIG. 13 shows the result of comparing such stress differences, and it can be seen that the sensor configured with three moduli exhibits a larger stress difference between the opposite side of the skin and the skin side than the sensor configured with the single modulus.

In this case, the smaller the tensile electrode angle, the lower the strain stress, and the larger the tensile electrode angle, the greater the strain stress may become.

For example, FIGS. 14 and 15 illustrate the difference in strain stress according to the tensile electrode angle, and it can be seen that the difference in strain stress occurs depending on the tensile direction and the tensile electrode angle. First, assuming that tension is applied in a pulling direction from both sides as illustrated in FIG. 14, it can be seen that the tensile electrode angle of −45 degrees exhibits the lowest strain stress distribution, while the tensile electrode angle of 45 degrees exhibits the highest strain stress distribution.

In this case, as the thickness of the sheet decreases, the initial capacitance value becomes smaller, but the sensitivity to capacitance change may be increased.

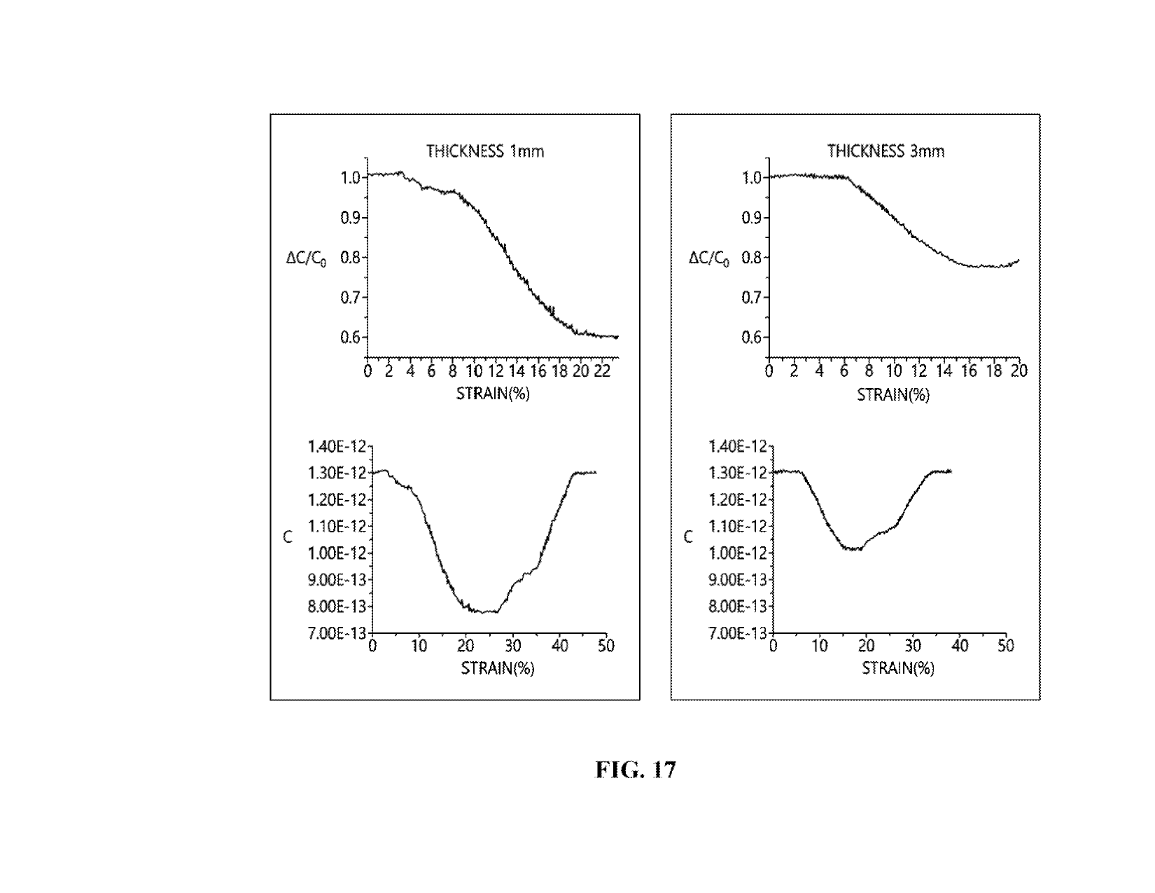

For example, assuming that the sensors are stretched at the same interval as illustrated in FIG. 16, the difference in capacitance between a sensor having an elastomer sheet with a thickness of 1 mm and a sensor having an elastomer sheet with a thickness of 3 mm may appear as illustrated in FIG. 17.

That is, it can be seen that as the thickness of the elastomer sheet decreases, the initial capacitance value becomes smaller, resulting in higher sensitivity to the same tensile length.

In this case, the capacitance sensitivity and hysteresis may be improved through elastomer material, doping with conductive material, or structural improvement.

In addition to the parameters described in detail, the characteristics of the sensor may be optimized by adjusting parameters such as the width W and length I of the flexible substrate, the number n of the films, and the length d excluding the overlapping area between the films.

By using the rolling strain sensor with the 3D structure, it is possible to provide a customized strain sensor according to specific conditions and requirements in application fields that require a miniaturized and lightweight sensor.

Further, the rolling strain sensor structure according to the present disclosure simplifies the manufacturing process from a 2D structure to a 3D structure, enables real-time detection of resistance or capacitance change corresponding to tension change to measure tension distribution, and can be manufactured to be advantageous for the application fields by optimizing diverse structural parameters.

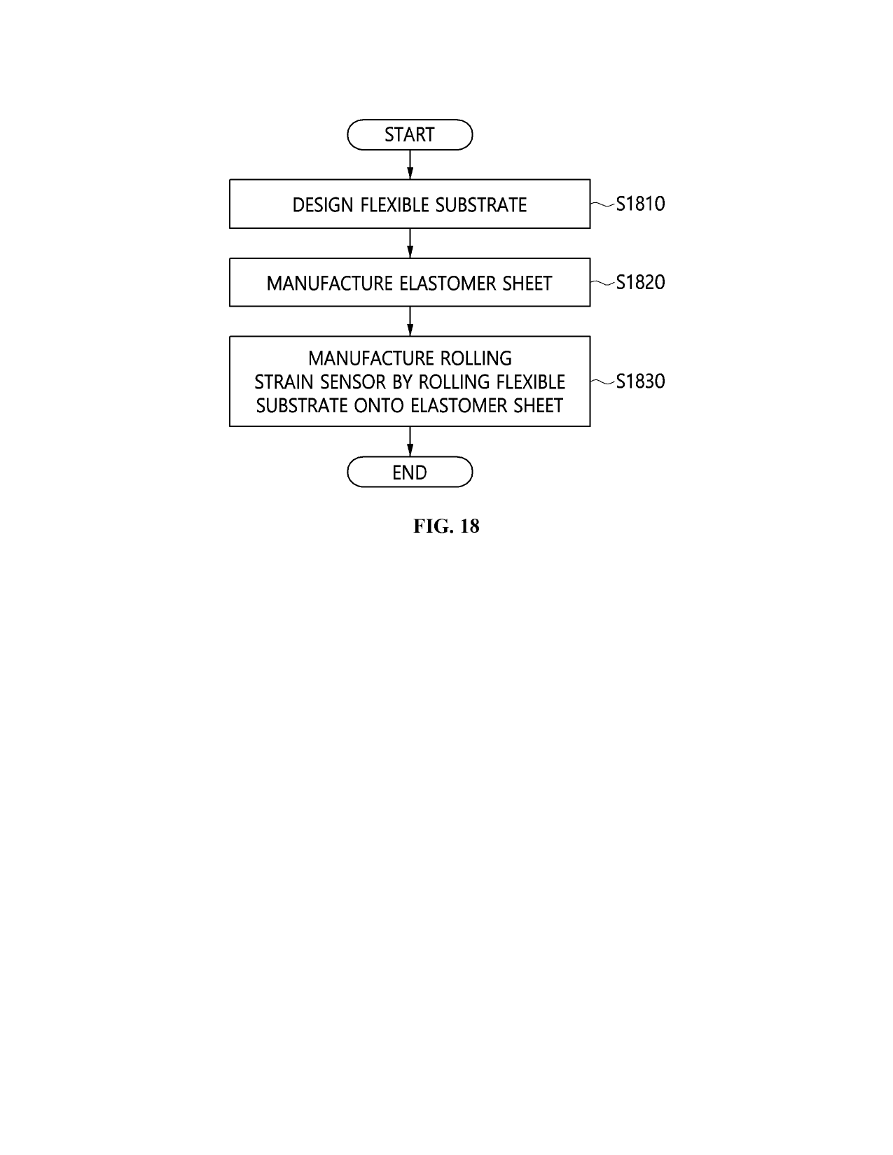

FIG. 18 is a flowchart illustrating a method of manufacturing a 3D-structured rolling strain sensor according to an embodiment of the present disclosure.

Referring to FIG. 18, the method of manufacturing the 3D-structured rolling strain sensor according to an embodiment of the present disclosure includes designing a flexible substrate by connecting multiple films having 2D electrode patterns formed thereon through tensile electrodes, at step S1810.

In this case, the flexible substrate may be designed in consideration of a first parameter corresponding to the width, length, number of the films, length excluding an overlapping area between the films, and a tensile electrode angle.

Further, the method of manufacturing the 3D-structured rolling strain sensor according to an embodiment of the present disclosure includes manufacturing at least one elastomer sheet using a stretchable material, at step S1820.

In this case, at least one elastomer sheet may be manufactured in consideration of a second parameter corresponding to the modulus, the thickness of the sheet, the number of sheets, and the arrangement method of the multiple sheets.

Further, the method of manufacturing the 3D-structured rolling strain sensor according to an embodiment of the present disclosure includes rolling the flexible substrate onto at least one elastomer sheet to manufacture the 3D-structured rolling strain sensor, at step S1830.

In this case, when an external strain is applied to the rolling strain sensor, the capacitance may change as the overlapping area between the upper and lower electrodes bonded to the at least one elastomer sheet varies.

In this case, the strain stress or the sensitivity to capacitance change of the rolling strain sensor may be adjusted based on the first parameter and the second parameter.

In this case, the rolling strain sensor may be configured such that the smaller the tensile electrode angle, the lower the strain stress, and the larger the tensile electrode angle, the greater the strain stress may become.

In this case, the rolling strain sensor may be configured such that, as the thickness of the sheet decreases, the initial capacitance value becomes smaller, but the sensitivity to capacitance change may be increased.

In this case, the rolling strain sensor may be configured such that, when at least one elastomer sheet is composed of multiple sheets having different moduli, strain stresses on the opposing surfaces in the sensor may differ from each other.

The method of manufacturing the 3D-structured rolling strain sensor can simplify a sensor manufacturing process and enables the mass production of the sensor, thereby greatly improving production efficiency.

According to the present disclosure, through a simple rolling manufacturing process, the process of manufacturing a 3D-structured rolling strain sensor can be simplified, and mass production can be achieved, thereby greatly improving production efficiency.

Further, the present disclosure can maintain consistent product quality by reducing errors that occur in a complex folding process of an existing origami process.

Furthermore, the present disclosure enables miniaturization and weight reduction through a design that minimizes the volume and weight of a strain sensor through structural optimization, and thus can be utilized in application fields such as a wearable device and a body-attachable sensor.

Furthermore, the present disclosure allows for the adjustment of various structural parameters, enabling the development of a customized strain sensor according to specific conditions and requirements.

As described above, in the rolling strain sensor and method of manufacturing the rolling strain sensor according to the present disclosure, the configurations and schemes in the above-described embodiments are not limitedly applied, and some or all of the above embodiments can be selectively combined and configured such that various modifications are possible.

What is claimed is:

1. A three-dimensional (3D)-structured rolling strain sensor, comprising:
a flexible substrate configured such that multiple films, each having a two-dimensional (2D) electrode pattern formed thereon, are connected through tensile electrodes; and
at least one elastomer sheet made of a stretchable material,
wherein the flexible substrate is bonded to form a 3D structure while being rolled onto the at least one elastomer sheet, and the 3D-structured rolling strain sensor is configured such that, when strain is externally applied, an overlapping area between upper and lower electrodes bonded to the at least one elastomer sheet varies, thereby changing a capacitance.

2. The 3D-structured rolling strain sensor of claim 1, wherein the flexible substrate is designed in consideration of a first parameter corresponding to a width, a length, a number of films, a length excluding an overlapping area between the films, and a tensile electrode angle.

3. The 3D-structured rolling strain sensor of claim 2, wherein the at least one elastomer sheet is manufactured in consideration of a second parameter corresponding to a modulus, a sheet thickness, a number of sheets, and an arrangement method for the multiple sheets.

4. The 3D-structured rolling strain sensor of claim 3, wherein a strain stress or sensitivity to capacitance change is adjusted based on the first parameter or the second parameter.

5. The 3D-structured rolling strain sensor of claim 4, wherein the strain stress decreases as the tensile electrode angle decreases, and increases as the tensile electrode angle increases.

6. The 3D-structured rolling strain sensor of claim 4, wherein, as the sheet thickness decreases, an initial capacitance value decreases, but the sensitivity to the capacitance change increases.

7. The 3D-structured rolling strain sensor of claim 4, wherein, when the at least one elastomer sheet is composed of multiple sheets having different moduli, strain stresses on facing surfaces in the sensor are different from each other.

8. A method of manufacturing a three-dimensional (3D)-structured rolling strain sensor, comprising:
designing a flexible substrate by connecting multiple films, each having a 2D electrode pattern formed thereon, through tensile electrodes;
manufacturing at least one elastomer sheet from a stretchable material; and
manufacturing a 3D-structured rolling strain sensor by rolling the flexible substrate onto the at least one elastomer sheet,
wherein the 3D-structured rolling strain sensor is configured such that, when strain is externally applied, an overlapping area between upper and lower electrodes bonded to the at least one elastomer sheet varies, thereby changing a capacitance.

9. The method of claim 8, wherein designing the flexible substrate comprises:
designing the flexible substrate in consideration of a first parameter corresponding to a width, a length, a number of films, a length excluding an overlapping area between the films, and a tensile electrode angle.

10. The method of claim 9, wherein manufacturing the at least one elastomer sheet comprises:
manufacturing the at least one elastomer sheet in consideration of a second parameter corresponding to a modulus, a sheet thickness, a number of sheets, and an arrangement method for the multiple sheets.

11. The method of claim 10, wherein a strain stress or sensitivity to capacitance change of the rolling strain sensor is adjusted based on the first parameter or the second parameter.

12. The method of claim 11, wherein the rolling strain sensor is configured such that the strain stress decreases as the tensile electrode angle decreases, and increases as the tensile electrode angle increases.

13. The method of claim 11, wherein the rolling strain sensor is configured such that, as the sheet thickness decreases, an initial capacitance value decreases, but the sensitivity to the capacitance change increases.

14. The method of claim 11, wherein the rolling strain sensor is configured such that, when the at least one elastomer sheet is composed of multiple sheets having different moduli, strain stresses on facing surfaces in the sensor are different from each other.
